(19) United States
(12) Patent Application Publication
(10) Pub. No.: US 2026/0000860 A1
(43) Pub. Date: Jan. 1, 2026

(54) IV SET RESTRAINT FOR STRAIN RELIEF AND PREVENTION OF ACCIDENTAL CATHETER REMOVAL

(71) Applicant: CareFusion 303, Inc., San Diego, CA (US)

(72) Inventors: Martin HOUSTON, Tralee (IE); Andrei REABOI, Limerick (IE); Michael Anthony GALVIN, Geashill (IE); Ronald HIDALGO, Pallaskenry (IE)

(21) Appl. No.: 18/754,938

(22) Filed: Jun. 26, 2024

(51) Int. Cl. A61M 25/00; A61M 5/14

(52) U.S. Cl. CPC A61M 25/002; A61M 5/1413; A61M 2025/0059; A61M 2205/0272

(57) ABSTRACT

An IV line storage device includes a housing and a cover. The housing includes an IV line inlet, a spool, and an IV line outlet. The spool is configured to receive an IV line such that a first segment of an IV line is wound around the spool. The spool has a predetermined diameter such that the spool and the housing define a gap with a predetermined width for receiving and storing the first segment of the IV line, and a predetermined height to accommodate the first segment of the IV line being wound around the spool a predetermined number of revolutions. The cover encloses the first segment of the IV line within the housing. The IV line storage device is configured to transfer strain incurred at a second segment of the IV line, outside of the housing, to the first segment of the IV line within the housing.

### FIELD OF THE INVENTION

The present disclosure generally relates to strain relief devices for an intravenous (IV) line, and, in particular, storage devices that are configured to store loose or exposed line between a medical device and a patient and absorb strain applied to the IV line before the strain reaches a patient.

### BACKGROUND

Intravenous (IV) administration sets include an IV line (e.g., a silicon tubing) that couples a fluid source or medical device to a patient. The IV line of an IV administration set, when not secured, can be pulled or snagged while a patient is engaged in everyday activities. The IV line, when pulled or snagged, can cause the IV administration set to be pulled from the patient and/or hurt the patient, which could lead to injury or infection. Additionally, the IV line, when pulled or snagged, can disrupt an infusion, and negatively affect the patient's treatment.

As such, there is a need for IV line storage devices that reduce or prevent an IV line from being snagged or pulled.

### SUMMARY

The disclosed subject matter relates to different IV strain relief devices (also referred to as IV line storage devices). The disclosed IV strain relief devices are configured to reduce an amount of exposed or loose IV line to prevent or reduce the risk of snags and/or pulls. Additionally, the disclosed IV strain relief devices are to absorb a strain and/or stress on the IV line incurred by an incidental snag and/or pull such that the strain and/or stress is not transferred to a patient. The disclosed IV strain relief devices allow users to easily manage IV line without disrupting their treatment (e.g., occluding flow of a medical fluid) and/or damaging the IV line. Additionally, the disclosed IV strain relief devices allow patients to store excess or loose IV line quickly and easily without requiring a high level of skill or force. The disclosed IV strain relief devices are configured to work with different types of IV administration sets, fluid sources, and/or medical devices and are reusable (e.g., can be coupled or decoupled from different lines non-destructively). Moreover, the disclosed IV strain relief devices can be easily assembled or disassembled and are safe for use while providing a cost-effective solution for managing an IV line.

In accordance with some implementations, an IV line storage device is provided. The IV line storage device includes a housing and a cover configured to couple with the housing. The housing includes an IV line inlet, a spool, and an IV line outlet. The spool is configured to receive an IV line such that a first segment of an IV line is wound around the spool. The spool has a predetermined diameter such that the spool and the housing define a gap with a predetermined width for receiving and storing the first segment of the IV line, and a predetermined height to accommodate the first segment of the IV line being wound around the spool a predetermined number of revolutions. The cover is configured to couple with the housing such that the first segment of the IV line is enclosed within the housing. Further, the IV line storage device is configured to transfer strain incurred at a second segment of the IV line, outside of the housing, to the first segment of the IV line within the housing.

In accordance with some implementations, a spring loaded IV line storage device is provided. The spring loaded IV line storage device includes a housing, a cover, a spool disposed within the housing, and a spring mechanism coupled with the spool. The spool includes a guide slot for receiving and securing a first segment of an IV line. The spring mechanism includes a spring element, a shaft, and an actuation element. The shaft is configured to rotate about an axis when a spring force is applied to or removed from the spring element. The actuation element, when actuated, applies or removes the spring force from the spring element. The spring mechanism is coupled with the spool via the shaft and the spool is caused to i) rotate about the axis when the spring force is applied to or removed from the spring element and ii) wind or unwind the first segment of the IV line around the spool when rotated about the axis. The cover includes an IV line inlet and an IV line outlet and is configured to couple with the housing such that the spool, the spring mechanism, and the first segment of the IV line are enclosed within the housing. The spring loaded IV line storage device is configured to reduce a second segment of the IV line that is outside of the housing while also providing strain/tension relief.

In accordance with some implementations, an IV line track device is provided. The IV line track device includes a track housing and a track cover configured to couple with the track housing. The track housing includes a track inlet, a track outlet, and a track path. The track path is within the housing and is configured to connect the track inlet and the track outlet, define an inner outline within the housing, and receive a first segment of an IV line. The tack cover, when coupled with the track housing encloses the first segment of the IV line within the housing. Further, the IV line track device is configured to release a sub-segment of the first segment of the IV line when a strain is incurred at a second segment of the IV line, outside of the track housing.

In accordance with some implementations, a system including an IV line storage device, a spring loaded IV line storage device, and/or an IV line track device is provided. The system includes a medical device or fluid source coupled to an IV administration set and at least one of the IV line storage device, the spring loaded IV line storage device, and/or the IV line track device coupled with an IV line of the IV administration set.

Note that the various implementations described above can be combined with other implementations described herein. The features and advantages described in the specification are not all inclusive and, in particular, additional features and advantages will be apparent to one of ordinary skill in the art in view of the drawings, specification, and claims. Moreover, it should be noted that the language used in the specification has been principally selected for readability and instructional purposes.

### BRIEF DESCRIPTION OF THE DRAWINGS

So that the present disclosure can be understood in greater detail, a more particular description may be had by reference to the features of various implementations, some of which are illustrated in the appended drawings. The appended drawings, however, merely illustrate pertinent features of the present disclosure. The description may admit to other effective features as the person of skill in this art will appreciate upon reading this disclosure.

FIGS. 1A-1C illustrate example strain relief devices for an IV administration set, in accordance with some implementations.

FIGS. 2A and 2B illustrate an IV line storage device, in accordance with some implementations.

FIGS. 3A and 3B illustrate an IV line track device, in accordance with some implementations.

FIGS. 4A-4C illustrate a spring loaded IV line storage device, in accordance with some implementations.

FIGS. 5A-5D illustrate different strain relief devices used together, in accordance with some implementations.

In accordance with common practice, the various features illustrated in the drawings are not drawn to scale. Accordingly, the dimensions of the various features may be arbitrarily expanded or reduced for clarity. In addition, some of the drawings may not depict all of the components of a given system, method or device. Finally, like reference numerals denote like features throughout the specification and figures.

### DETAILED DESCRIPTION

Numerous details are described herein in order to provide a thorough understanding of the example implementations illustrated in the accompanying drawings. However, some implementations may be practiced without many of the specific details, and the scope of the claims is only limited by those features and aspects specifically recited in the claims. Furthermore, well-known processes, components, and materials have not been described in exhaustive detail so as to avoid obscuring pertinent aspects of the implementations described herein.

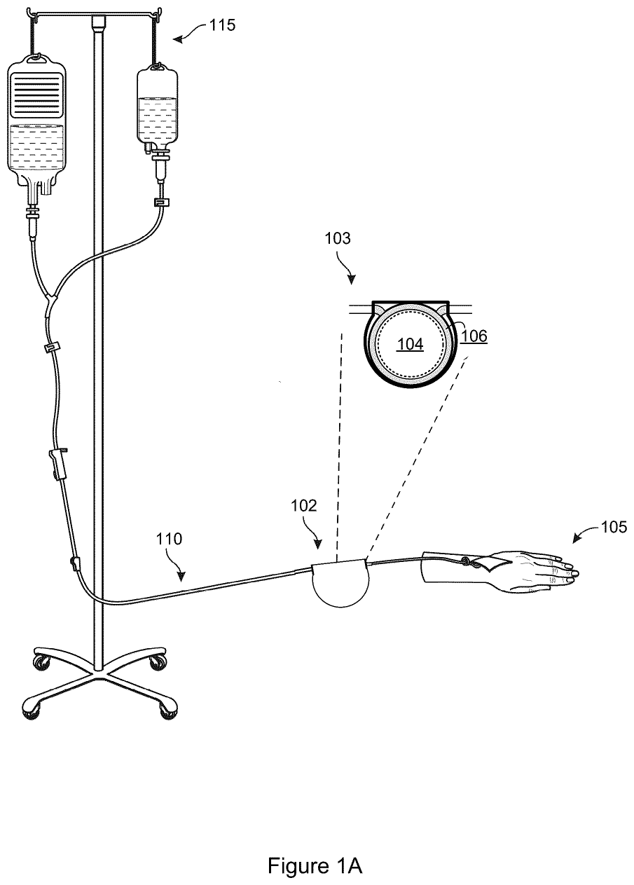

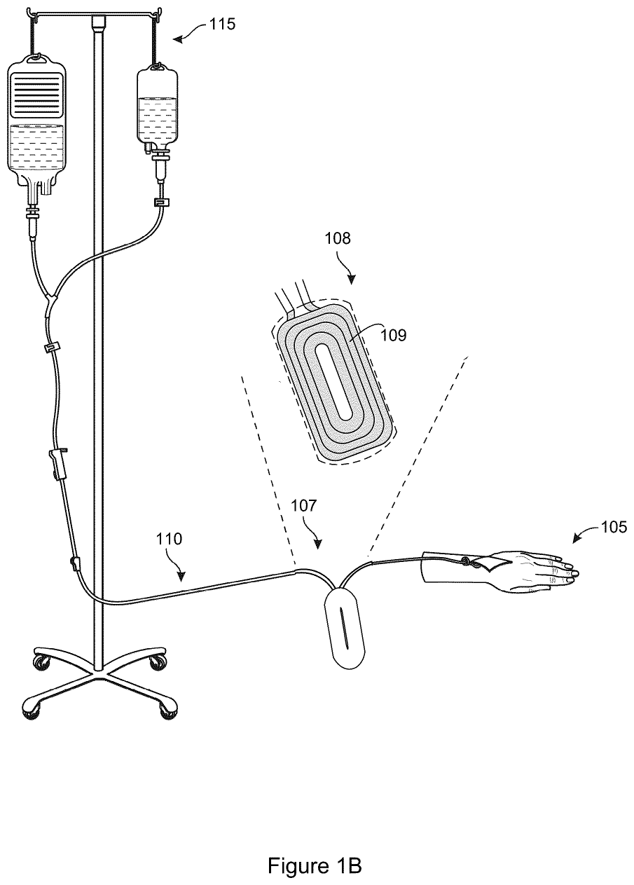

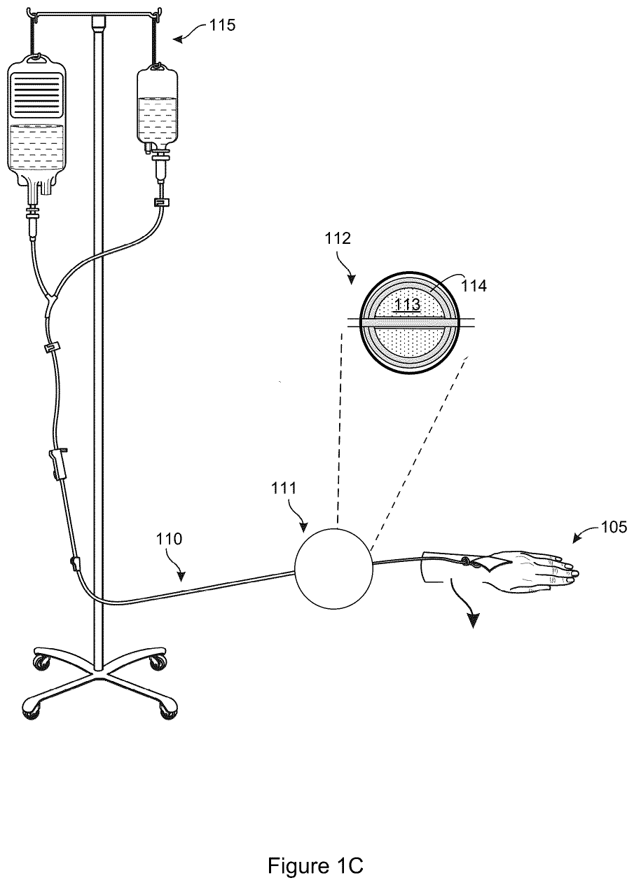

FIGS. 1A-1C illustrate example strain relief devices for an IV administration set, in accordance with some implementations. FIGS. 1A-1C show an IV administration set 110 coupled to a patient 105 (e.g., via an IV line or tubing). The IV administration set 110 includes a fluid source 115 and is configured to allow for a flow of a fluid, such as a medical fluid, from the fluid source 115 to the patient 115. In some implementations, the IV administration set 110 is coupled to a medical device, such as an infusion device (e.g., IV pump), for administering a medical fluid to a patient 105. The IV pump is configured to administer the medical fluid to the patient at a predetermined and uniform rate. The IV administration set 110 can include excess tubing (or an IV line) that can be pulled or tugged while coupled to the patient 105 (e.g., excess line can snag on objects, get caught by trolley wheels or corners of countertops, get stood or sat on, etc.), which can lead to complications such as the IV line being pulled out or the infusion being slowed or stopped.

The example strain relief devices disclosed herein are configured to couple with an IV line of an IV administration set. Specifically, the example strain relief devices are configured to receive a segment the IV line of an IV administration set to reduce excess segments of the IV line (e.g., unsecured segments of the IV line that can snag or be incidentally pulled). Moreover, the example strain relief devices are configured to reduce and/or eliminate strain transferred to a patient using the IV administration set when an excess segment of the IV line (or other segment line) is snagged or pulled. The example strain relief devices are configured to be coupled to lines of any IV administration set without restricting a patient's movements or affecting infusion (e.g., disrupting a flow of fluid to the patient). Additionally, the example strain relief devices are configured to couple or decouple from an IV administration set non-destructively (e.g., without damaging the IV line or the example strain relief devices), such that the example strain relief devices can be reused on any IV administration set.

In FIG. 1A, a first example strain relief device 102 is shown. The first example strain relief device 102 is configured to receive and couple with a segment of the IV line of the IV administration set 110. In particular, an excess portion of the IV line of the IV administration set 110 is configured to be wound within an interior portion 103 of the first example strain relief device 102. The interior portion 103 of the first example strain relief device 102 includes a spool 104 or drum that is configured to receive a segment of the IV line (e.g., wound line 106).

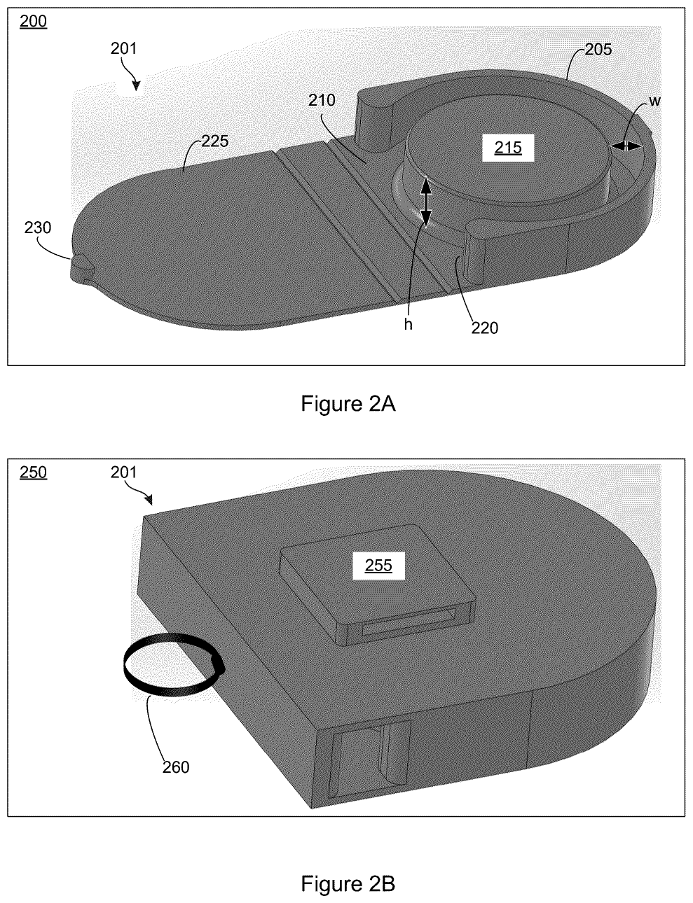

The spool 104 is fixed and configured to receive a predetermined length of IV line. More specifically, the spool 104 allows for the IV line to be wound a predetermined number of revolutions (e.g., at least two revolutions). When a cover of the first example strain relief device 102 encloses the wound line 106, the wound line 106 is held in place via friction (e.g., friction between overlapping portions of the IV line and/or friction between the wound line 106 and the housing of the first example strain relief device 102. When strain (e.g., caused by snagging or pulling on the IV line) is experienced on segments of the IV line that are external to the device 102, the first example strain relief device 102 is configured to transfer the strain to the wound line 106 such that the strain is reduced or eliminated before reaching the patient 105. The first example strain relief device 102 is described in detail below in reference to FIGS. 2A and 2B.

FIG. 1B shows a second example strain relief device 107. The second example strain relief device 108 is configured to receive and couple with a segment of the IV line of the IV administration set 110. In particular, an excess segment of the IV line of the IV administration set 110 is configured to be disposed within a within an interior track portion 108 of the second example strain relief device 107. The interior track portion 108 of the second example strain relief device 107 includes a track path or channel that defines an inner outline of a housing of the second example strain relief device 107. In some implementations, the inner outline forms one or more concentric patterns. For example, as shown in FIG. 1B, a received a segment of the IV line (e.g., wrapped line 109) is disposed within the interior track portion 108 of the second example strain relief device 107 (e.g., the track path) and forms concentric ellipses or circles.

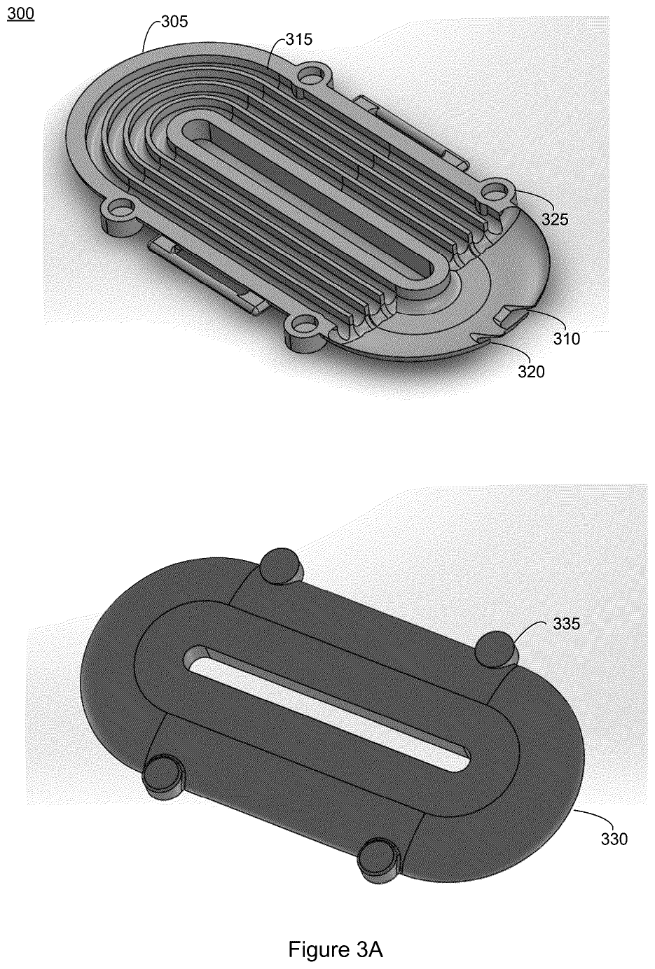

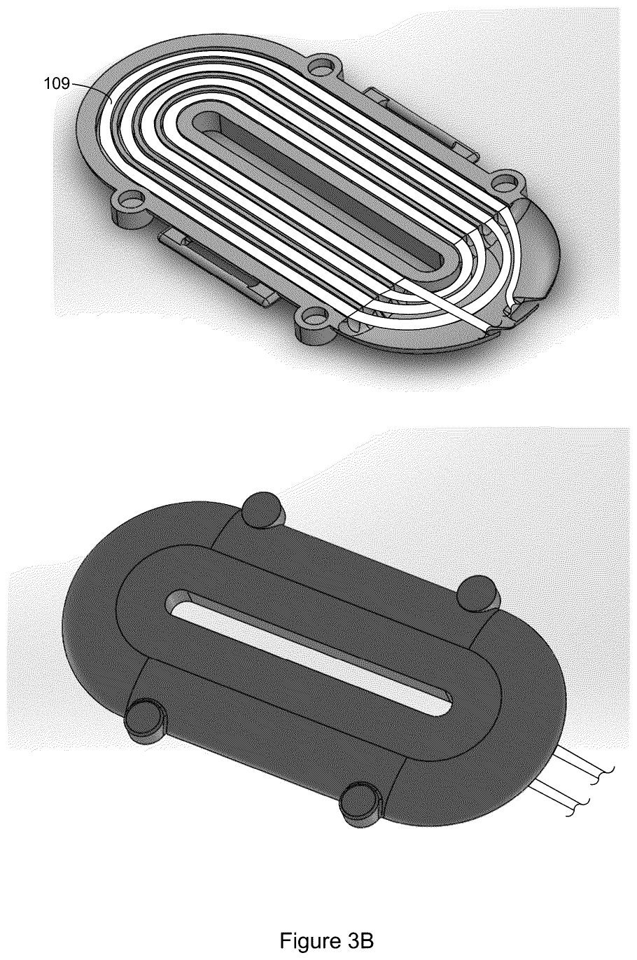

The track path is configured to receive a predetermined length of IV line and to allow for at least a segment of the received IV line to be released from the second example strain relief device 107 when an external segment of the IV line outside of the second example strain relief device 107 is strained beyond a threshold tension (e.g., 50 newtons, 75 newtons, 100 newtons, etc.). More specifically, the second example strain relief device 107 is configured to release stored line (e.g., one row/channel at a time) when an exposed segment of the IV line is pulled or snagged. By releasing the stored line, the second example strain relief device 107 increases the exposed length of the IV line of the IV administration set 110 to reduce or eliminate a strain felt by the patent. For example, when an exposed segment of the IV line is strained (e.g., snagged or pulled), the wrapped line 109 starts to become undone, which increases the length of the IV line and allows the patient to take appropriate action before the strain is transferred to them (e.g., the patient can remove the source of the snag or pull that is releasing the wrapped line 109 from the second example strain relief device 107 before the snag or pull is felt at patient's body). The second example strain relief device 107 further includes a cover for enclosing the wrapped line 109 within a housing of the second example strain relief device 107. The second example strain relief device 107 is described in detail below in reference to FIGS. 3A-3C.

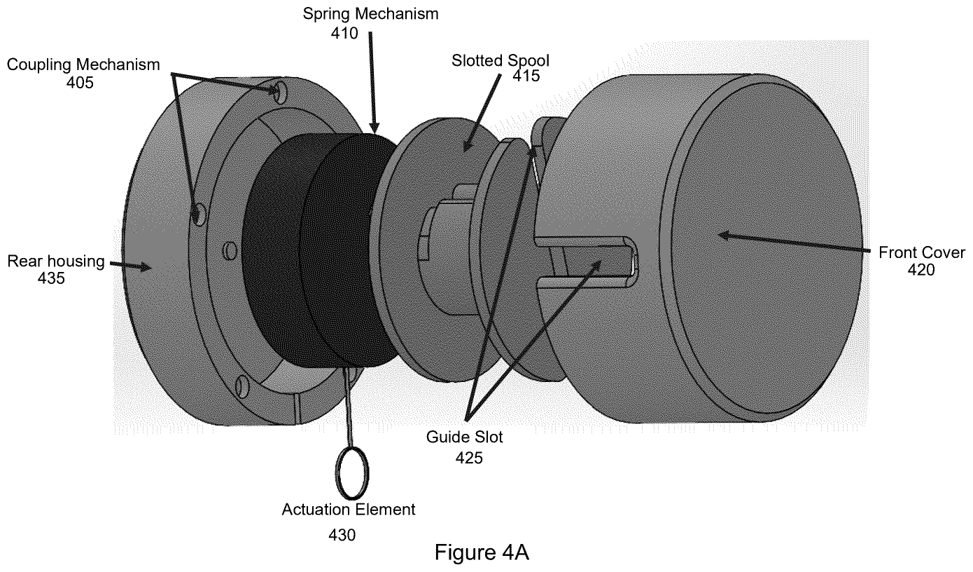

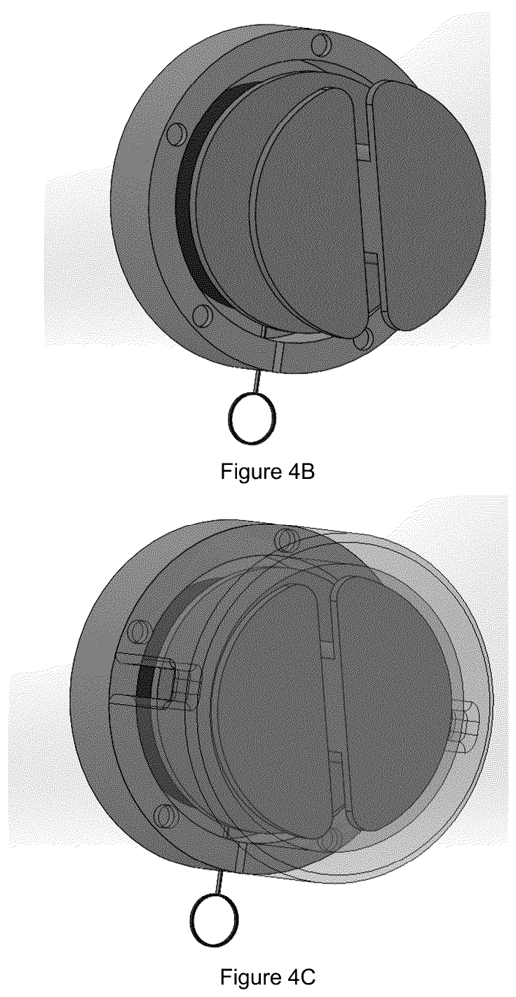

A third example strain relief device 111 is shown in FIG. 1C. The third example strain relief device 111 is configured to receive and couple with a segment of the IV line of the IV administration set 110. In particular, an excess segment of the IV line of the IV administration set 110 is configured to be mechanically wound within an interior casing 112 of the third example strain relief device 111. The interior casing 112 of the third example strain relief device 111 includes a slotted spool 113 or slotted drum and a spring mechanism 410 (FIG. 4A, e.g., a spring-loaded reel). The slotted spool 113 is configured to receive a segment of the IV line and the spring mechanism 410 is configured to wind the segment of the IV line around the slotted spool 113 (e.g., forming coiled line 114).

The third example strain relief device 111 includes actuation element 430 (FIG. 4A) for winding or unwinding the spring mechanism 410 such that a segment of the IV line is wound on or unwound from the slotted spool 113 (when the actuation element 430 is actuated). Similar to the first and second example strain relief devices 102 and 107, the third example strain relief device 111 is configured to reduce or eliminate the risk of the (exposed) line from being snagged on objects (e.g., countertops, stands, etc.) and/or being stepped on or otherwise kinked. The third example strain relief device 111 is further configured to prevent a segment of the IV line from touching or trailing on floor and provides a more aesthetically pleasing appearance (e.g., a neater and tidier appearance).

A front casing 420 (FIG. 4A) is configured to couple with the interior casing 112. The front casing 420 includes one or more guide slots and is configured to work in conjunction with the slotted spool 113 and the spring mechanism 410 to wind or unwind the IV line. The third example strain relief device 111 is described in detail below in reference to FIGS. 4A-4C.

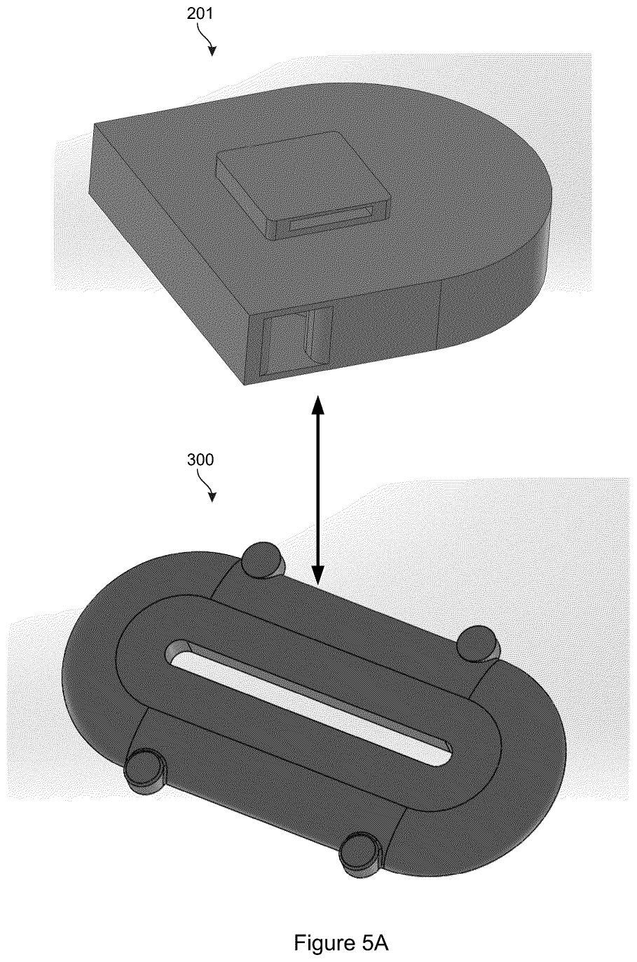

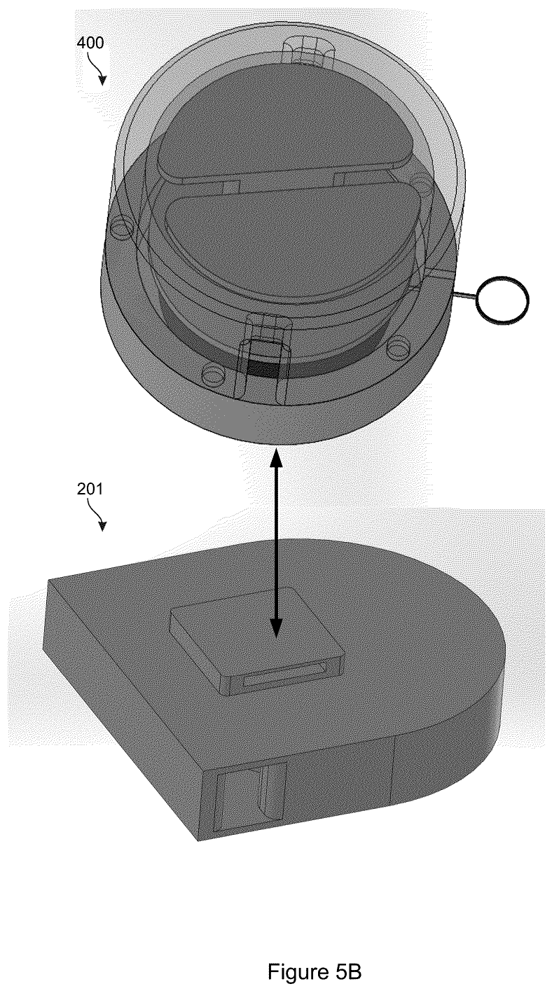

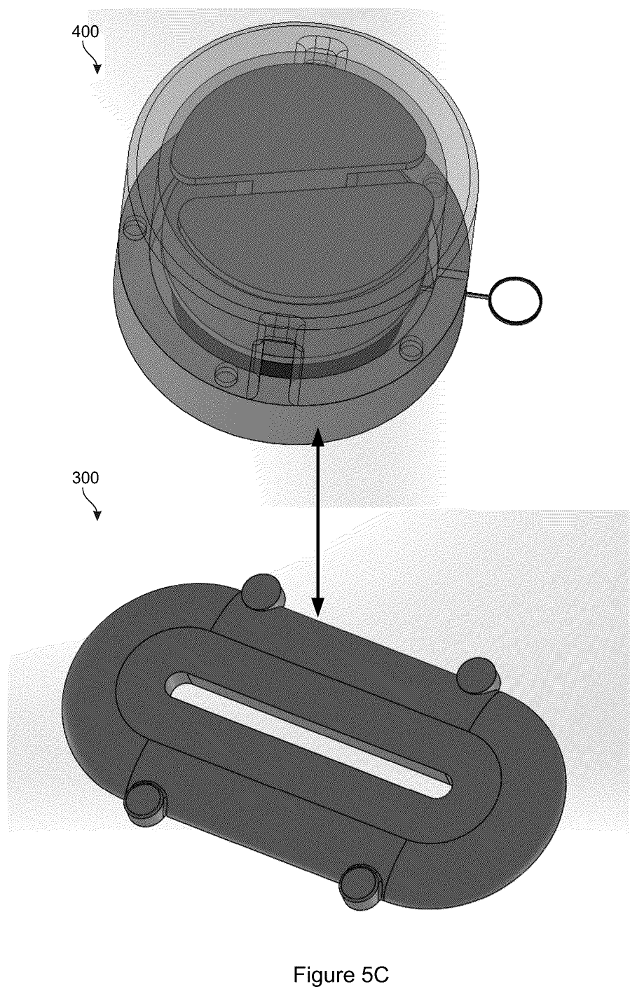

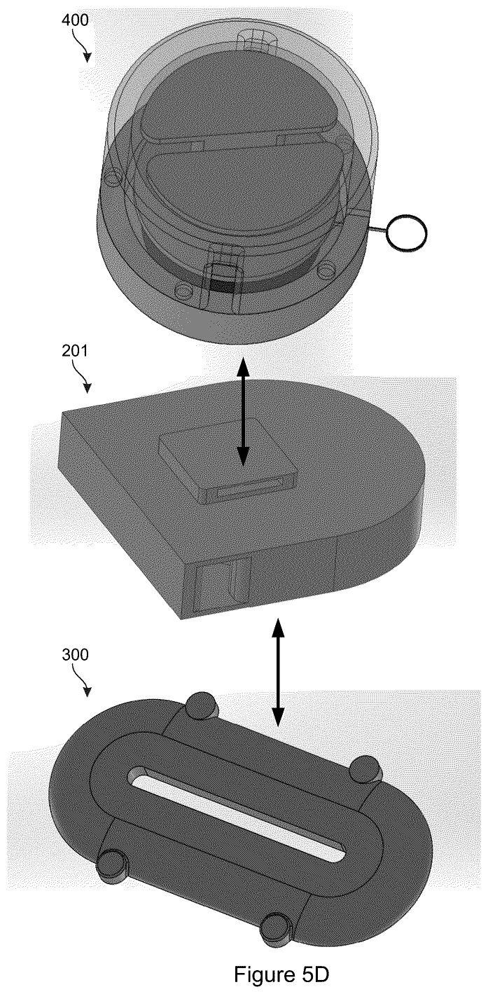

While FIGS. 1A-1C show the example strain relief devices used individually, in some implementations, the different strain relief devices can be together. The different strain relief devices can be coupled in series and/or coupled on top of one another (as shown and described below in reference to FIGS. 5A-5D.

FIGS. 2A and 2B illustrate an IV line storage device, in accordance with some implementations. FIG. 2A shows an open configuration 200 of the IV line storage device 201 and FIG. 2B shows a closed configuration 250 of the IV line storage device 201. The IV line storage device 201 is an instance of the first example strain relief device 102 described above in refence to FIG. 1A. The IV line storage device 201 includes a housing 205, an IV line inlet 210, a spool 215 (or drum), an IV line outlet 220, and a cover 225. In some implementations, the housing 205, the spool 215, the IV line inlet 210, the IV line outlet 220, and the cover 225 are unitary component. In some implementations, the IV line storage device 201 is a molded component, 3D printed component, or machined component. The IV line storage device 201 is configured to transfer strain incurred at a second segment of the IV line, outside of the housing 205, to a first segment of the IV line within the housing 205 as described below.

The spool 215 (analogous to spool 104; FIG. 1A) is configured to receive an IV line of an IV administration set such that a first segment of an IV line is wound around the spool 215. The spool 215 has a predetermined diameter such that the spool 215 and the housing 205 define a gap with a predetermined width (w) for receiving the first segment of the IV line. The spool 215 further has a predetermined height (h) such that the first segment of the IV line is configured to be wound around the spool a predetermined number of revolutions. In some implementations, the predetermined number of revolutions is at least two. In some implementations, the predetermined width of the gap is less than two times a diameter of the IV line. In some implementations, the predetermined height of the spool is two times a diameter of the IV line or less that two times a diameter of the IV line. In this way, the spool 215 allows for the first segment of the IV line to include overlapping portions that generate friction at the first segment of the IV line.

The cover 225 is configured to couple with the housing 205 such that the first segment of the IV line is enclosed within the housing 205. In some implementations, the cover 225 includes a coupling mechanism 230 for coupling the cover 225 to the housing 205. In some implementations, the coupling mechanism 230 is a magnet, a clasp, a hook, etc. The cover 225, when coupled to the housing 205, maintains a friction generated by the overlapping portions of the first segment of the IV line. In particular, the cover 225, when coupled to the housing 205, does not allow the first segment of the IV line to be unwound or decoupled from the spool 215 such that the friction between overlapping portions of the IV line is maintained. In some implementations, the friction between overlapping portions of the first segment of the IV line limit movement of the IV line and/or absorb a portion (or all) of the strain before it is felt by a patient. In some implementations, a material of the IV line storage device 201 is selected based on a desired friction (e.g., between the spool 215, the housing 205, and the cover and the IV line).

In some implementations, the IV line inlet and line outlet include rounded edges. The rounded edges reduce or eliminate a risk of an infusion being disrupted. Specifically, the round edges allow for smart routing that ensures a low risk of occlusion.

The IV line storage device 201 is configured to be manually loaded. For example, while in the open configuration 200, a patient is able two wind the IV line at least twice around the spool 215. After the winding the IV line, the patient can close the cover 225. In some implementations, a spring mechanism can be included in the IV line storage device 201 to allow the patient to quickly wind the IV line around the spool 215.

The closed configuration 250 of the IV line storage device 201 shows an outer portion of the housing 205. In implementations, the IV line storage device 201 includes an IV line coupling fixture 255 and a carrying fixture 260.

The IV line coupling fixture 255 allows the patient to store excess line. In particular, the IV line coupling fixture 255 allows the patient to use a strap, a hook-and-loop fastener, or other fastener to attach a second segment of the IV line (e.g., an exposed segment of the IV line) to the IV line storage device 201. Alternatively, or in addition, an IV line track device 300 (FIGS. 3A-3C) and/or a spring loaded IV line storage device (FIGS. 4A-4C) can be coupled to the IV line storage device 201 to store excess line.

The carrying fixture 260 allows the patient to couple the IV line storage device 201 to their person. In some implementations, the carrying fixture 260 is a keychain, a belt loop, a carabiner, or other similar device that allows the patient to couple the IV line storage device 201 to their body and/or clothing.

FIGS. 3A and 3B illustrate an IV line track device, in accordance with some implementations. FIG. 3A shows different portions of the IV line track device 300 and FIG. 3B show an IV line disposed within the IV line track device 300. The IV line track device 300 is an instance of the second example strain relief device 107 described above in refence to FIG. 1B. The IV line track device 300 includes a track housing 305, a track inlet 310, a track path 315, a track outlet 320, a first set of coupling mechanisms 325, a track cover 330, and a second set of coupling mechanisms 335. In some implementations, the IV line track device 300 is molded, 3D printed, or machined. The IV line track device 300 is configured to release a sub-segment of a first segment of the IV line (disposed within the track path 315) when a strain is incurred at a second segment of the IV line, outside of the track housing as described below.

The track path 315 is disposed within and defines a channel within the track housing 305. The channel of the track path 315 is configured to receive a first segment of an IV line of an IV administration set. Each track within the track path 315 has a width substantially equal to a diameter of the IV line. For example, where the IV line consists of a 5 mm silicon tubing (and, e.g., 3 mm internal diameter), each track may be 5 mm wide. In some implementations, the track may be less than 5 mm or greater than 5 mm by a few microns, depending on how much tension (e.g., in newtons) is desirable to release the IV line from the track(s) when tension is applied. The track path 315 defines an inner outline within the track housing 305. In some implementations, the inner outline forms one or more concentric patterns. For example, as shown in FIG. 3A, the track path 315 defines an inner outline within the track housing 305 that form concentric ovals. The track path 315 further connects the track inlet 310 and the track outlet 320. The track path 315 allows the first segment of the IV line to be housed within the track housing 305 such that there is less excess or exposed line. When a strain is applied to exposed line, the track path 315 is configured to release a sub-segment of the first segment of the IV line increasing the exposed length of the IV line and limiting a strain transferred to a patient. Additionally, by releasing a sub-segment of the first segment of the IV line, the patient is provided with a predetermined amount of time to remove the source of the strain (if present).

The track housing 305 further includes a first set of coupling mechanism 325. The first set of coupling mechanism 325 are used to couple the track housing 305 to the track cover 330. In some implementations, the first set of coupling mechanism 325 are magnets, claps, hooks, or other coupling devices.

The track cover 330 is configured to couple with the track housing 305 such that the first segment of the IV line is enclosed within the track housing 305. In some implementations, the track cover 330 includes a symmetrical track path 315 such that, when the track cover 330 is coupled with the track housing 305, the first segment of the IV line is disposed with the channel (e.g., without being occluded or kinked).

The track cover 330 can include a second set of coupling mechanisms 335. The second set of coupling mechanism 335 couple the track cover 330 to the track housing 305. In some implementations, the second set of coupling mechanism 335 couple with the first set of coupling mechanism 325. For example, in some implementations, the second set of coupling mechanism 335 can be a magnetic surface configured to coupled with magnets of the first set of coupling mechanism 325, and vice versa. In some implementations, the second set of coupling mechanism 335, like the first set of coupling mechanism 325, are magnets, claps, hooks, or other coupling devices.

The IV line track device 300 is configured to be manually loaded. For example, while the track cover 330 is removed, a patient is able to place a segment of the IV line within the track path 315, with respective segments of the IV line entering and exiting within the track inlet 310 and track outlet 320. In some implementations, the IV line track device 300 includes a carrying fixture (not shown) that allows the patient to couple the IV line track device 300 to their person (e.g., their body and/or clothing).

In some implementations, the IV line track device 300 is configured to couple with and IV line storage device 201 (FIGS. 2A and 2B) and/or a spring loaded IV line storage device (FIGS. 4A-4C).

FIG. 3B shows an IV line of an IV administration set disposed within the IV line track device 300. As shown in FIG. 3B, the IV line enters via the track inlet 310, is disposed within and follows the track path 315, and exits via the track outlet 320.

FIGS. 4A-4C illustrate a spring loaded IV line storage device, in accordance with some implementations. FIG. 4A shows an explode view of the spring loaded IV line storage device 400. The spring loaded IV line storage device 400 is an instance of the third example strain relief device 111 described above in refence to FIG. 1C. The spring loaded IV line storage device 400 includes a rear housing 435 (e.g., back casing), one or more guide slots 425, a slotted spool 415 (or slotted drum), a spring mechanism 410, an actuation element 430, and a front cover 420 (e.g., front casing). In some implementations, the spring loaded IV line storage device 400 includes one or more coupling mechanisms 405 for coupling the back casing 435 to the front casing 420. In some implementations, the coupling mechanisms 405 are magnets, claps, hooks, or other coupling devices. In some implementations, a portion of the spring loaded IV line storage device 400 is molded, 3D printed, and/or machined. The spring loaded IV line storage device 400 is configured to reduce a second segment of the IV line, outside of the spring loaded IV line storage device 400 (e.g., reduce the total exposed line of an IV administration set) as described below.

The spring mechanism 410 (e.g., a spring-loaded reel) is coupled to the slotted spool 415 and, when actuated, causes the slotted spool 415 to rotate in a clockwise or counterclockwise direction, which winds or unwinds the IV line around the slotted spool 415. The spring mechanism 410 is actuated via an actuation element 430. The actuation element 430 can be a hand or finger member (e.g., finger ring) coupled to a cord and configured to be pulled a predetermined distance such that the spring mechanism 410 is loaded with a spring force (based on the distance at which the actuation element 430 is pulled). Further, when the actuation element 430 is pulled, the spring mechanism 410 rotates the slotted spool 415 a first direction (clockwise or counterclockwise). The actuation element 430 can be held by the user or locked in place at the predetermined distance while the user loads the IV line onto the slotted spool 415 as discussed below. When the actuation element 430 is released by the user or freed from a locked position, the spring force of the spring mechanism 410 is released, which causes the spring mechanism 410 to rotate the slotted spool 415 a second direction (opposite the first direction) and wind the IV line around the slotted spool 415. To unwind and remove the IV line from the slotted spool 415, the user can actuate the actuation element 430 a subsequent time and remove the IV line while the spring force on the spring mechanism 410 is maintained (e.g., the actuation element 430 is held by the user or locked in place at the predetermined distance).

In some embodiments, the spring mechanism 410 includes a spring element (not show), a shaft (not shown), and the actuation element 430. The shaft is configured to rotate about an axis when a spring force is applied to or removed from the spring element. The actuation element 430, as described above, applies or removes the spring force from the spring element when actuated. The spring mechanism 410 is coupled with the slotted spool 415 (of spool 104) via the shaft. The slotted spool 415 is caused to i) rotate about the axis when the spring force is applied to or removed from the spring element and ii) wind or unwind the first segment of the IV line around the spool when rotated about the axis. In some embodiments, the slotted spool 415 (of spool 104) is coupled directly onto a (rotating) planar surface of the spring mechanism 410. While the spring mechanism 410 is describes as being manually actuated by the actuation element 430, in some implementations the spring mechanism 410 is automatically actuated by one or more motors (not shown).

The slotted spool 415 (analogous to slotted spool 113; FIG. 1C) includes a guide slot and is configured to receive an IV line of an IV administration set such that a first segment of an IV line can be wound around the slotted spool 415. The slotted spool 415 is configured to secure the first segment of the IV line when wound. For example, as described above, when the spring mechanism 410 is actuated and held in place (e.g., such that the spring mechanism 410 has a spring force) the user can secure the IV line around the slotted spool 415. When the spring force on the spring mechanism 410 is released (e.g., the actuation element 430 is released), the slotted spool 415, which is coupled with the spring mechanism 410, is caused to rotate. When the slotted spool 415 is rotated by the spring mechanism 410, the first segment of the IV line is wound or unwound from the slotted spool 415. The slotted spool 415 and the spring mechanism 410 are coupled to the rear housing 435.

The front cover 420 is configured to couple with rear housing 435 such that the slotted spool 415, the spring mechanism 410, and the first segment of the IV line are enclosed within the spring loaded IV line storage device 400. The front cover 420 includes one or more guide slots 425 that are used to wind and unwind the first segment of the IV line (e.g., wrap or unwrap the IV line around the spool when the slotted spool 415 is actuated via the spring mechanism 410). In some implementations, the one or more guide slots 425 on the front cover 420 are configured to operate as an IV line inlet and line outlet.

The spring loaded IV line storage device 400 allows a patient to store excess line of an IV administration set quickly and efficiently. By storing excess line, the spring loaded IV line storage device 400 holds the stored IV line with a predetermined tension (e.g., a spring force provided by the spring mechanism 410, such as 50 newtons, 75 newtons, 100 newtons, etc.) such that, when a strain (or a force opposite to the spring force) is experience at exposed segments of the IV line, the spring mechanism 410 releases a segment of the stored IV line and relieves a strain on the IV line. Additionally, the spring loaded IV line storage device 400 is configured to reduces or eliminates the risk of occlusion (e.g., kinks or steps on the IV line), line dragging on the floor or snagging on objects, and/or minimizes hazards.

The spring loaded IV line storage device 400 is configured to be substantially automatically loaded. In particular, the IV line of an IV administration set needs to be disposed within the slotted spool 415 and the front cover 420 needs to be coupled to the rear housing 435 (after the IV line is received by the slotted spool 415) before the actuation element 430 can be actuated. The patient can wind and unwind the IV line by actuating the actuation element 430. The patient can manually remove the IV line from the spring loaded IV line storage device 400.

In some implementations, the spring loaded IV line storage device 400 is configured to couple with and IV line storage device 201 (FIGS. 2A and 2B) and/or an IV line track device 300 (FIGS. 3A and 3B).

FIG. 4B shows the spring loaded IV line storage device 400 with the front cover 420 removed.

FIG. 4C shows the spring loaded IV line storage device 400 with the front cover 420 attached (but transparent).

FIGS. 5A-5D illustrate different strain relief devices used together, in accordance with some implementations.

FIG. 5A illustrates an IV line storage device 201 used in conjunction with an IV line track device 300. In some implementations, the IV line storage device 201 is coupled to the IV line track device 300. In some implementations, the IV line storage device 201 and the IV line track device 300 are couple to an IV line of an IV administration set in series.

FIG. 5B illustrates an IV line storage device 201 used in conjunction with a spring loaded IV line storage device 400. In some implementations, the IV line storage device 201 is coupled to the spring loaded IV line storage device 400. In some implementations, the IV line storage device 201 and the spring loaded IV line storage device 400 are couple to an IV line of an IV administration set in series.

FIG. 5C illustrates an IV line track device 300 used in conjunction with a spring loaded IV line storage device 400. In some implementations, the IV line track device 300 is coupled to the spring loaded IV line storage device 400. In some implementations, the IV line track device 300 and the spring loaded IV line storage device 400 are couple to an IV line of an IV administration set in series.

FIG. 5D illustrates an IV line storage device 201 used in conjunction with an IV line track device 300 and a spring loaded IV line storage device 400. In some implementations, the IV line storage device 201 is coupled to the IV line track device 300 and the spring loaded IV line storage device 400 (e.g., sandwiched between the two devices). In some implementations, the IV line storage device 201, the IV line track device 300 and the spring loaded IV line storage device 400 are couple to an IV line of an IV administration set in series. Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. An IV line storage device. The IV line storage device includes a housing and a cover configured to couple with the housing. The housing includes an IV line inlet, a spool, and an IV line outlet. The spool is configured to receive an IV line such that a first segment of an IV line is wound around the spool. The spool has a predetermined diameter such that the spool and the housing define a gap with a predetermined width for receiving and storing the first segment of the IV line, and a predetermined height to accommodate the first segment of the IV line being wound around the spool a predetermined number of revolutions. The cover is configured to couple with the housing such that the first segment of the IV line is enclosed within the housing. Further, the spool transfers strain incurred at a second segment of the IV line, outside of the housing, to the first segment of the IV line within the housing.
Clause 2. The IV line storage device of Clause 1, the first segment of the IV line includes overlapping portions of the IV line that generate friction at the first segment of the IV line, and the cover, when coupled to the housing, maintains the friction generated by the overlapping portions of the IV line.
Clause 3. The IV line storage device of any of Clause 1 through Clause 2, the IV line inlet and the IV line outlet include rounded edges.
Clause 4. The IV line storage device of any of Clause 1 through Clause 3, the predetermined number of revolutions is at least two.
Clause 5. The IV line storage device of any of Clause 1 through Clause 4, the predetermined width of the gap is less than two times a diameter of the IV line.
Clause 6. The IV line storage device of any of Clause 1 through Clause 5, the predetermined height of the spool is two times a diameter of the IV line or less that two times a diameter of the IV line.
Clause 7. The IV line storage device of any of Clause 1 through Clause 6, the housing, the spool, and the cover are a unitary component.
Clause 8. The IV line storage device of any of Clause 1 through Clause 7, further including a spring mechanism including a spring element, a shaft, and an actuation element. The shaft is configured to rotate about an axis when a spring force is applied to or removed from the spring element. The actuation element, when actuated, applies or removes the spring force from the spring element. The spring mechanism is coupled with the spool via the shaft and the spool is caused to i) rotate about the axis when the spring force is applied to or removed from the spring element and ii) wind or unwind the first segment of the IV line around the spool when rotated about the axis.
Clause 9. The IV line storage device of any of Clause 1 through Clause 8, the IV line storage device is coupled with a spring loaded IV line storage device of any of Clause 11 through Clause 13 or an IV line track device of any of Clause 16 through Clause 18.
Clause 10. The IV line storage device of Clause 9, the IV line storage device is coupled with a segment of IV line downstream and/or upstream from the spring loaded IV line storage device and/or coupled with a segment of IV line downstream and/or upstream from the IV line track device.
Clause 11. A spring loaded IV line storage device. The spring loaded IV line storage device includes a housing, a cover, a spool disposed within the housing, and a spring mechanism coupled with the spool. The spool includes a guide slot for receiving and securing a first segment of an IV line. The spring mechanism includes a spring element, a shaft, and an actuation element. The shaft is configured to rotate about an axis when a spring force is applied to or removed from the spring element. The actuation element, when actuated, applies or removes the spring force from the spring element. The spring mechanism is coupled with the spool via the shaft and the spool is caused to i) rotate about the axis when the spring force is applied to or removed from the spring element and ii) wind or unwind the first segment of the IV line around the spool when rotated about the axis. The cover includes an IV line inlet and an IV line outlet and is configured to couple with the housing such that the spool, the spring mechanism, and the first segment of the IV line are enclosed within the housing. The spring loaded IV line storage device is configured to reduce a length of a second segment of the IV line that is outside of the housing.
Clause 12. The spring loaded IV line storage device of Clause 11, the cover is configured to couple with the housing via one or more magnets.
Clause 13. The spring loaded IV line storage device of any of Clause 11 through Clause 12, the IV line inlet and the IV line outlet of the cover are configured to operate as additional guide slots such that, when the actuation element is actuated, the first segment of the IV line is wound on or unwound from the spool without entangling.
Clause 14. The spring loaded IV line storage device of any of Clause 11 through Clause 13, the spring loaded IV line storage device is coupled with an IV line storage device of Clause 1 through Clause 8 or an IV line track device of any of Clause 16 through Clause 18.
Clause 15. The spring loaded IV line storage device of Clause 14, the spring loaded IV line storage device is coupled with a segment of IV line downstream and/or upstream from the IV line storage device and/or coupled with a segment of IV line downstream and/or upstream from the IV line track device.
Clause 16. An IV line track device. The IV line track device includes a track housing and a track cover configured to couple with the track housing. The track housing includes a track inlet, a track outlet, and a track path. The track path is within the housing and is configured to connect the track inlet and the track outlet, define an inner outline within the housing, and receive a first segment of an IV line. The tack cover, when coupled with the track housing encloses the first segment of the IV line within the housing. Further, the track housing releases a sub-segment of the first segment of the IV line when a strain is incurred at a second segment of the IV line, outside of the track housing.
Clause 17. The IV line track device of Clause 16, the inner outline is a forms one or more concentric patterns.
Clause 18. The IV line track device of any of Clause 16 through Clause 17, the track cover is configured to couple with the track housing via one or more magnets.
Clause 19. The IV line track device of any of Clause 16 through Clause 18, the IV line track device is configured to couple with an IV line storage device of Clause 1 through Clause 8 and/or a spring loaded IV line storage device of any of Clause 11 through Clause 13.
Clause 18. The IV line track device of Clause 17, the IV line track device is coupled with a segment of IV line downstream and/or upstream from the IV line storage device and/or a segment of IV line downstream and/or upstream from the spring loaded IV line storage device.
Clause 19. A system including a medical device, an IV administration set couple with the medical device, and at least one of i) an IV line storage device of Clause 1 through Clause 10, ii) a spring loaded IV line storage device of Clause 11 through Clause 15, and/or iii) an IV line track device of any of Clause 16 through Clause 18. The IV line storage device, the spring loaded IV line storage device, and/or the IV line track device coupled to a segment of the IV line of the IV administration set.
Clause 20. The system of Clause 18, the medical device is an infusion device or a fluid source.
Clause 21. The system of any of Clause 19 through Clause 20, the IV line storage device, the spring loaded IV line storage device, and/or the IV line track device are coupled downstream from the medical device.
Clause 22. The system of any of Clause 19 through Clause 21, the IV line storage device, the spring loaded IV line storage device, and/or the IV line track device are configured to couple to a user's person.
Clause 23. The system of any of Clause 19 through Clause 22, the IV line storage device, the spring loaded IV line storage device, and/or the IV line track device are configured to be non-destructively decoupled from the IV line of the IV administration set.

### Further Consideration:

In some implementations, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

It will be understood that, although the terms “first,” “second,” etc. may be used herein to describe various elements, these elements should not be limited by these terms. These terms are only used to distinguish one element from another. Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

The terminology used herein is for the purpose of describing particular implementations only and is not intended to be limiting of the claims. As used in the description of the implementations and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such an implementation may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Various items may be arranged differently (e.g., arranged in a different order, or partitioned in a different way) all without departing from the scope of the subject technology.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various implementations for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) set line storage device, comprising:
a housing including:
an IV line inlet,
a spool configured to receive an IV line such that a first segment of the IV line is wound around the spool, wherein the spool has:
a predetermined diameter such that the spool and the housing define a gap with a predetermined width for receiving and storing the first segment of the IV line, and
a predetermined height to accommodate the first segment of the IV line being wound around the spool a predetermined number of revolutions, and
an IV line outlet;
a cover configured to couple with the housing such that the first segment of the IV line is enclosed within the housing; and
wherein the spool transfers strain incurred at a second segment of the IV line, outside of the housing, to the first segment of the IV line within the housing.

2. The IV line storage device of claim 1, wherein:
the first segment of the IV line includes overlapping portions of the IV line that generate friction at the first segment of the IV line; and
the cover, when coupled to the housing, maintains the friction generated by the overlapping portions of the IV line.

3. The IV line storage device of claim 1, wherein the IV line inlet and the IV line outlet include rounded edges.

4. The IV line storage device of claim 1, wherein the predetermined number of revolutions is at least two.

5. The IV line storage device of claim 1, wherein the predetermined width of the gap is less than two times a diameter of the IV line.

6. The IV line storage device of claim 1, wherein the predetermined height of the spool is two times a diameter of the IV line or less that two times a diameter of the IV line.

7. The IV line storage device of claim 1, wherein the housing, the spool, and the cover are a unitary component.

8. The IV line storage device of claim 1, wherein the IV line storage device is configured to couple to an IV line track device, the IV line track device comprising:
a track housing including:
a track inlet,
a track outlet, and
a track path within the housing, wherein the track path is configured to:
connect the track inlet and the track outlet,
define an inner outline within the housing, and
receive a third segment of an IV line;
a track cover configured to couple with the track housing such that the third segment of the IV line is enclosed within the housing; and
wherein the track housing releases a sub-segment of the third segment of the IV line when a strain is incurred at a fourth segment of the IV line, outside of the track housing.

9. The IV line storage device of claim 8, wherein the IV line track device is coupled with a segment of the IV line downstream or upstream from the IV line storage device.

10. The IV line storage device of claim 1, wherein the IV line storage device is configured to couple to a spring loaded IV line storage device, the spring loaded IV line storage device comprising:
another housing;
another spool disposed within the housing, the other spool including a guide slot for receiving and securing a fifth segment of the IV line;
a spring mechanism coupled with the other spool, the spring mechanism including:
a spring element,
a shaft configured to rotate about an axis when a spring force is applied to or removed from the spring element, and
an actuation element for applying or removing the spring force from the spring element when actuated;
another cover including another IV line inlet and another IV line outlet, the other cover configured to couple with the other housing such that the other spool, the spring mechanism, and the fifth segment of the IV line are enclosed within the other housing; and
wherein:
the spring mechanism is coupled with the other spool via the shaft,
the other spool is caused to i) rotate about the axis when the spring force is applied to or removed from the spring element and ii) wind or unwind the fifth segment of the IV line around the other spool when rotated about the axis, and
the spring loaded IV line storage device is configured to reduce a length of a sixth segment of the IV line, outside of the other housing.

11. The IV line storage device of claim 10, wherein the spring loaded IV line storage device is coupled with a segment of the IV line downstream or upstream from the IV line storage device.

12. A spring loaded IV line storage device, comprising:
a housing;
a spool disposed within the housing, the spool including a guide slot for receiving and securing a first segment of an IV line;
a spring mechanism coupled with the spool, the spring mechanism including:
a spring element,
a shaft configured to rotate about an axis when a spring force is applied to or removed from the spring element, and
an actuation element for applying or removing the spring force from the spring element when actuated;
a cover including an IV line inlet and an IV line outlet, the cover configured to couple with the housing such that the spool, the spring mechanism, and the first segment of the IV line are enclosed within the housing; and
wherein:
the spring mechanism is coupled with the spool via the shaft,
the spool is caused to i) rotate about the axis when the spring force is applied to or removed from the spring element and ii) wind or unwind the first segment of the IV line around the spool when rotated about the axis, and
the spring loaded IV line storage device is configured to reduce a length of a second segment of the IV line, outside of the housing.

13. The spring loaded IV line storage device of claim 12, wherein the cover is configured to couple with the housing via one or more magnets.

14. The spring loaded IV line storage device of claim 12, wherein the IV line inlet and the IV line outlet of the cover are configured to operate as additional guide slots such that, when the actuation element is actuated, the first segment of the IV line is wound on or unwound from the spool without entangling.

15. The spring loaded IV line storage device of claim 12, wherein the spring loaded IV line storage device is configured to couple to an IV line track device, the IV line track device comprising:
a track housing including:
a track inlet,
a track outlet, and
a track path within the housing, wherein the track path is configured to:
connect the track inlet and the track outlet,
define an inner outline within the housing, and
receive a third segment of an IV line;
a track cover configured to couple with the track housing such that the third segment of the IV line is enclosed within the housing; and
wherein the track housing releases a sub-segment of the third segment of the IV line when a strain is incurred at a fourth segment of the IV line, outside of the track housing.

16. An IV line track device, comprising:
a track housing including:
a track inlet,
a track outlet, and
a track path within the housing, wherein the track path is configured to:
connect the track inlet and the track outlet,
define an inner outline within the housing, and
receive a first segment of an IV line;
a track cover configured to couple with the track housing such that the first segment of the IV line is enclosed within the housing; and
wherein the track housing releases a sub-segment of the first segment of the IV line when a strain is incurred at a second segment of the IV line, outside of the track housing.

17. The IV line track device of claim 16, wherein the inner outline is a forms one or more concentric patterns.

18. The IV line track device of claim 16, wherein the track cover is configured to couple with the track housing via one or more magnets.

19. The IV line track device of claim 16, wherein the IV line track device is configured to couple to a spring loaded IV line storage device, the spring loaded IV line storage device comprising:
a housing;
a spool disposed within the housing, the spool including a guide slot for receiving and securing a third segment of the IV line;
a spring mechanism coupled with the other spool, the spring mechanism including:
a spring element,
a shaft configured to rotate about an axis when a spring force is applied to or removed from the spring element, and
an actuation element for applying or removing the spring force from the spring element when actuated;
a cover including an IV line inlet and an IV line outlet, the cover configured to couple with the housing such that the spool, the spring mechanism, and the third segment of the IV line are enclosed within the housing; and
wherein:
the spring mechanism is coupled with the other spool via the shaft,
the other spool is caused to i) rotate about the axis when the spring force is applied to or removed from the spring element and ii) wind or unwind the third segment of the IV line around the other spool when rotated about the axis, and
the spring loaded IV line storage device is configured to reduce a length of a fourth segment of the IV line, outside of the housing.

20. The IV line track device of claim 19, wherein the spring loaded IV line storage device is coupled with a segment of the IV line downstream or upstream from the IV line track device.
